(19) United States
(12) Patent Application Publication
(10) Pub. No.: US 2019/0030039 A1
(43) Pub. Date: Jan. 31, 2019

(54) PREVENTION OF SSRI-INDUCED GASTROINTESTINAL DYSFUNCTION WITH A 5-HT4 RECEPTOR ANTAGONIST

(71) Applicant: The Trustees of Columbia University in the City of New York, New York, NY (US)

(72) Inventors: Kara Gross Margolis, New York, NY (US); Michael David Gershon, New York, NY (US)

(21) Appl. No.: 16/068,317

(22) PCT Filed: Jan. 20, 2017
(86) PCT No.: PCT/US17/14394

§ 371 Date: Jul. 5, 2018

Related U.S. Application Data
(60) Provisional application No. 62/281,484, filed on Jan. 21, 2016.

(51) Int. Cl. A61K 31/5365; A61P 1/00; A61P 39/02

(52) U.S. Cl. CPC A61K 31/5365; A61P 39/02; A61P 1/00

(57) ABSTRACT

The present invention provides, inter alia, methods for treating or preventing a disorder in a subject exposed to an agent that antagonizes the serotonin reuptake transporter (SERT), such as an SSRI, in utero or by consumption by the subject of breast milk from the subject's mother, by administering a 5-HT4 receptor antagonist, for example, to the subject's mother.

### GOVERNMENT FUNDING

This invention was made with government support under grant nos. KO8DK093786 and RO1NS015547 awarded by the National Institutes of Health. The government has certain rights in the invention.

### BACKGROUND OF THE INVENTION

Depression occurs in 14-23% of pregnant women. Selective serotonin reuptake inhibitors (SSRIs), e.g., Fluoxetine/Prozac, are the most widely prescribed medications used to treat this depression. SSRI use has increased from 1.5% to 6.4% of all U.S. pregnancies over the last decade. SSRIs cross the placenta and are present in the breast milk. At birth, plasma Fluoxetine levels in newborns are approximately 70% of maternal levels. Further, SSRI exposure in utero is associated with a two-fold increased risk of congenital malformations and alterations in central nervous system (CNS) development.

SSRIs act by blocking the serotonin reuptake transporter (SERT), the primary transporter responsible for serotonin inactivation. The inhibition of SERT with SSRIs thus inhibits serotonin inactivation leading to an increase in serotonergic neurotransmission. SERT and serotonin are present in both the CNS and the intestine. During central nervous system (CNS) development, serotonin is neurogenic and plays a key role in many aspects of brain development. As in the CNS, intestinal serotonin is neurogenic and critically modulates enteric nervous system (ENS) development and key gastrointestinal (GI) functions including intestinal motility, peristalsis, intestinal epithelial permeability, and predisposition to intestinal inflammatory disease. Abnormalities in serotonin levels lead to dysfunction in both CNS and ENS development and function. The CNS findings in human and rodent studies are similar; studies evaluating the effects of in utero SSRI exposure on CNS development reveal persistent changes in brain circuitry leading to maladaptive behaviors such as anxiety and depression that persist into adulthood. In contrast to the vast data available on the CNS, and despite the ample data implicating serotonin (5-HT) in ENS development and GI function, there has been only one study evaluating the effects of gestational SSRI exposure on ENS development. The findings, however, are impressive: children exposed to SSRIs in utero were 10 times more likely to be treated for constipation than unexposed children, suggesting that in utero exposure to SSRIs produce long-lasting effects on ENS development and GI motility.

Recently, SSRIs have indeed been shown to have a profound, long-lasting impact on ENS development and function. Murine progeny exposed to SSRIs in utero and during breastfeeding exhibit a profound hyperplasia of the ENS and defects in critical GI functions including motility, peristalsis, intestinal epithelial permeability and predisposition to intestinal inflammatory disease. Further, these defects that occur during development persist into adulthood. Similar intestinal abnormalities have been found in SERT knockout mice, a congenital model of chronic SSRI exposure, implicating the role of SERT antagonism in these abnormalities and not off-target drug effects of SSRls. Thus, SERT antagonism by SSRI exposure during neurodevelopment critically affects both brain and intestinal development, leading to long-lasting changes in GI function.

### SUMMARY OF THE INVENTION

With strong data to support the notion that antagonism of SERT by SSRIs causes significant abnormalities in ENS and CNS development, GI function, and behavior (i.e., anxiety and depression), a need exists to find a way to correct these abnormalities. The present invention is directed to overcoming the gastrointestinal and behavioral deficiencies noted above. Accordingly, in some embodiments of the present invention, there is provided a method of treating or preventing a disorder in a subject in need thereof, wherein the disorder is caused by in utero exposure and/or exposure through consumption of the subject's mother's breast milk to one or more agents that antagonize the serotonin reuptake transporter (SERT). This method comprises administering an effective amount of a 5-HT4 receptor antagonist to the subject.

In other embodiments of the present invention, there is provided a method of reducing the risk of developing a disorder in a subject, wherein the subject has been exposed in utero to a serotonin reuptake transporter (SERT) antagonist. This method comprises administering a therapeutically effective amount of a 5-HT4 receptor antagonist to the subject's mother during pregnancy and/or breastfeeding.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a bar graph showing total intestinal transit time (expressed as percentage of control). Maternal dams were administered either water (control), Fluoxetine (SSRI), or both Fluoxetine and Piboserod (SSRI+Pibo) from embryonic day 1 (E1) through weaning from breastfeeding. Pups were thus exposed to aforementioned medications during gestation and breastfeeding. Once pups were weaned, at postnatal day 21 (P21), they were not given any medications. Pups were then evaluated at 6-8 weeks of age to rule out acute drug effects. Total intestinal transit time was measured by administering a bolus of Rhodamine B (time 0) and calculating the amount of time until the rhodamine was found in the stool.

FIG. 2 is a bar graph showing colonic transit time (expressed as percentage of control). Maternal dams were administered either water (control), Fluoxetine (SSRI), or both Fluoxetine and Piboserod (SSRI+Pibo) from E1 through weaning from breastfeeding. Pups were thus exposed to aforementioned medications during gestation and breastfeeding. Once pups were weaned, at P21, they were not given any medications. Pups were evaluated at 6-8 weeks of age to rule out acute drug effects. Colonic transit time was measured by inserting a 2 mm glass bead 4 cm into the rectum and calculating the time it took for bead expulsion to occur.

FIGS. 3A-3D are a series of bar graphs showing results from the novelty-suppressed feeding (NSF) test, a measurement of anxiety. Maternal dams were administered either water (control), Fluoxetine (SSRI), or both Fluoxetine and Piboserod (SSRI+Pibo) from E1 through weaning from breastfeeding. Pups were thus exposed to aforementioned medications during gestation and breastfeeding. Once pups were weaned, at P21, they were not given any medications. The pups were evaluated at 6-8 weeks of age to rule out acute drug effects. The NSF test began with a short term food restriction of 18 hours (water was still available). After the test, mice were given food ad libitum again. The NSF test is a “conflict” test, which assesses the competing motivations of the animal to explore (forage) for food versus remain in the safety of the periphery of the arena. Mice were placed into a white box with shavings on the floor and two food pellets placed on a circle of filter paper in the center of the arena. The latency to chew the food is the measure of anxiety. Measured times included time to reach the filter paper with the food pellet on it (“time to filter”), time to touch/smell the pellet (“time to pellet”), time to bite the pellet (“time to bite”) and time to sit and eat the pellet (“time to sit and eat”).

FIG. 4 is a bar graph showing results from the sucrose preference test, a measurement of depression in mice. Maternal dams were administered either water (control), Fluoxetine (SSRI), or both Fluoxetine and Piboserod (SSRI+Pibo) from E1 through weaning from breastfeeding. Pups were thus exposed to aforementioned medications during gestation and breastfeeding. Once pups were weaned, at P21, they were not given any medications. The pups were evaluated at 6-8 weeks of age to rule out acute drug effects. For the sucrose preference test, mice were trained for 2 days to drink diluted sucrose and water from serological pipettes with sippers attached and testing was done on day 3. The volumes of sucrose and water consumed overnight were recorded. A reduced preference for sucrose solution over plain water represents anhedonia.

### DETAILED DESCRIPTION OF THE INVENTION

The subject matter of the present invention relates to methods of treating or preventing a disorder in a subject in need thereof, wherein the disorder is caused by in utero exposure or exposure through the subject's mother's breast milk to one or more agents that antagonize the serotonin reuptake transporter (SERT). The method includes administering an effective amount of a 5-HT4 receptor antagonist to the subject via the subject's mother.

As used herein, the terms “treat,” “treating,” “treatment” and grammatical variations thereof mean exposing an individual subject to a protocol, regimen, process or remedy, in which it is desired to obtain a physiologic response or outcome in that subject, e.g., a patient. However, because every treated subject may not respond to a particular treatment protocol, regimen, process or remedy, treating or preventing does not require that the desired physiologic response or outcome be achieved in each and every subject or subject population, e.g., patient population. Accordingly, a given subject or subject population, e.g., patient population, may fail to respond or respond inadequately to treatment. In the present invention, the subject may be in utero or being fed through the subject's mother's breast milk. Thus, as set forth in more detail below, the treatment is provided to the subject's mother in a manner such that effective treatment in the subject is achieved.

As used herein, the terms “prevent,” “preventing,” or “prevention,” and grammatical variations thereof refer to the prophylactic treatment of a subject in need thereof.

As used herein, the term “disorder” broadly refers to a syndrome, condition, chronic illness or a particular disease. For example, the disorder may be a gastrointestinal disorder or a behavioral disorder. In the present invention, a “gastrointestinal disorder” refers to a disease involving the gastrointestinal tract, namely the esophagus, stomach, small intestine, large intestine and rectum, and the accessory organs of digestion, the liver, gallbladder, and pancreas. Thus, as used herein, “gastrointestinal disorder” includes but is not limited to: constipation, intestinal inflammatory disease, irritable bowel syndrome, enteric nervous system hyperplasia, Crohn's disease, ulcerative colitis, microscopic colitis, and combinations thereof. The disorder may also be a disorder of the CNS caused by exposure of the subject to an agent, such as an SSRI, while in utero and/or during breastfeeding. Other non-limiting examples of disorders according to the present invention include: an anxiety disorder, a mood disorder, a depressive disorder, an autism spectrum disorder, a substance abuse or dependence disorder, an attention deficit hyperactivity disorder, and combinations thereof.

In some aspects of this and other embodiments of the invention, the subject is a mammal. Preferably, the mammal is selected from the group consisting of humans, primates, farm animals, and companion animals such as dogs and cats. More preferably, the mammal is a human. The term does not denote a particular age. Thus, both adult and newborn animals, as well as fetuses, are intended to be covered.

As used herein, the term “antagonist” means a compound or composition that reduces, interferes with, or inhibits physiological activity. For example, an antagonist of the serotonin reuptake transporter (SERT) prevents the removal of serotonin from the synaptic cleft by the transporter. In the present invention, an “agent” means a compound or composition that antagonizes SERT. Non-limiting examples of agents of the invention include: selective serotonin reuptake inhibitors (SSRIs), serotonin-norepinephrine reuptake inhibitors (SNRIs), trycyclic antidepressants (TCAs), atypical antidepressants, and combinations thereof. Preferably, the agent is an SSRI, such as, e.g., citalopram (Celexa; Forest Labs), escitalopram (Lexapro; Forest Labs), fluoxetine (Prozac; Eli Lilly), fluvoxamine (Luvox; Abbott Laboratories), fluvoxamine CR (Luvox CR; Jazz Pharmaceuticals), paroxetine (Paxil; GlaxoSmithKline), paroxetine CR (Paxil CR; GlaxoSmithKline), and sertraline (Zoloft; Pfizer).

In the present invention, a “5-HT4 receptor antagonist” is any drug or other composition which binds to the 5-HT4 receptor as defined by the IUPHAR (see Pharmacological Reviews, 1994, 44, 157-203) and prevents the activation of the 5-HT4 receptor. As used herein, the 5-HT4 receptor antagonist may be selective for 5-HT4 receptors or it may be non-selective, exhibiting agonist or antagonist activity at other serotonin receptors. Preferably, the 5-HT4 receptor antagonist is selective for 5-HT4 receptors. Non-limiting examples of 5-HT4 receptor antagonists include: Piboserod (GlaxoSmithKline), indisetron (Nisshin Pharma), fabesetron (Astellas), sulamserod (Roche), E-2630 (Eisai), GR-113,808 (GlaxoSmithKline), GR-125,487 (GlaxoSmithKline), LY-297524 (Eli Lilly), M-0014 (Shire), R-216073 (Johnson & Johnson), RO-1160367 (Roche), RS-23597-190 (Tocris), RS-39604 (Tocris), SB-203,186 (GlaxoSmithKline), SB-204,070 (GlaxoSmithKline), SB-205,008 (GlaxoSmithKline), SB-205,800 (GlaxoSmithKline), SB-207,710 (GlaxoSmithKline), SER-103 (Serodus), SER-120 (Serodus), chamomile (ethanol extract), SDZ 205-557 (Novartis), SC-53606 (Sigma), and combinations thereof.

The terms “administering”, “administration” and variants thereof (particularly “administering” an agent or antagonist) as used herein means introducing an agent, e.g., a 5-HT4 receptor antagonist, into the body of a subject, such as a human, in need of such treatment. In some embodiments, the 5-HT4 receptor antagonist is administered to the subject indirectly; specifically, the antagonist is administered to the subject's mother while the subject is in utero. In some embodiments, the 5-HT4 receptor antagonist is administered concomitantly with the one or more agents that antagonize SERT. In some embodiments, the 5-HT4 receptor antagonist is administered perinatally and/or postnatally, directly to the subject. In other embodiments, the 5-HT4 receptor antagonist is administered perinatally and/or postnatally to the subject's mother, and indirectly to the subject across the placental barrier or via breast milk. Thus, when the subject is in utero, administration of the agent to the mother must be in a form that crosses the placental barrier, must not interfere with any treatment regimen (e.g. SSRI therapy) of the mother, and must be safe for both the mother and the fetus. When the subject is breast feeding, administration of the agent to the mother must be in a form that passes into the mother's breast milk, must not interfere with any treatment regimen (e.g. SSRI therapy) of the mother, and must be safe for both the mother and the subject.

In the present invention, an “effective amount” or a “therapeutically effective amount” of an agent, such as a 5-HT4 receptor antagonist, is an amount that is sufficient to effect beneficial or desired results as described herein when administered to a subject directly or indirectly via the subject's mother. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of any active agent disclosed herein will be that amount of the active agent which is the lowest dose effective to produce the desired effect.

The following examples are provided to further illustrate certain aspects of the present invention. These examples are illustrative only and are not intended to limit the scope of the invention in any way.

### EXAMPLES

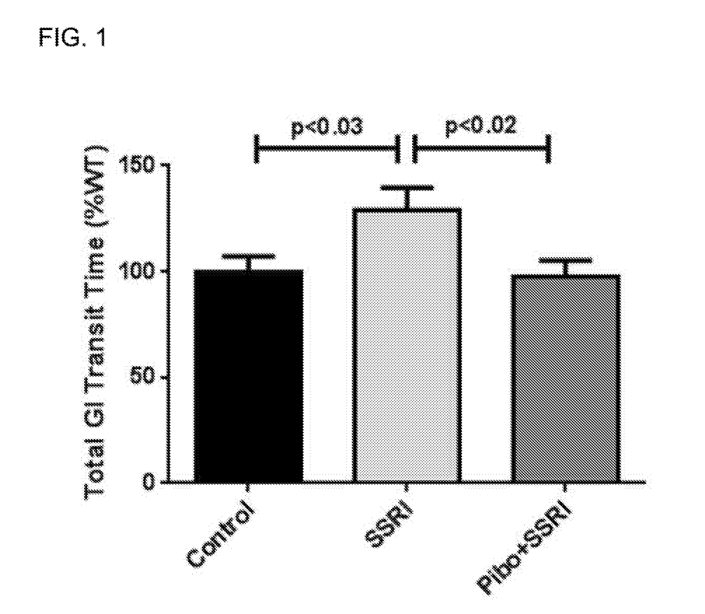

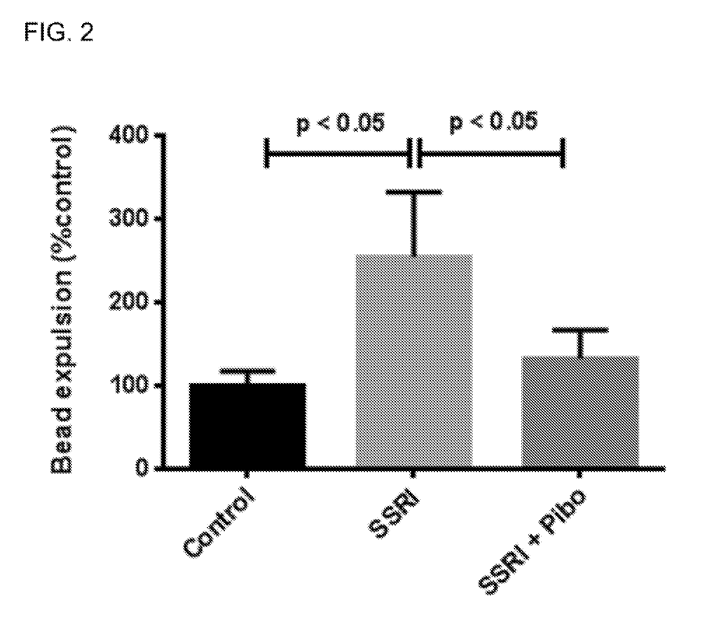

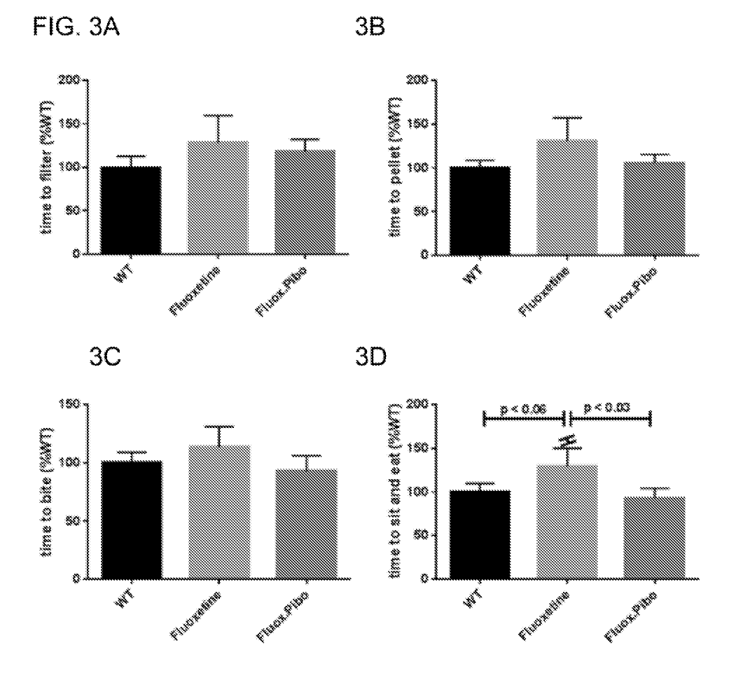

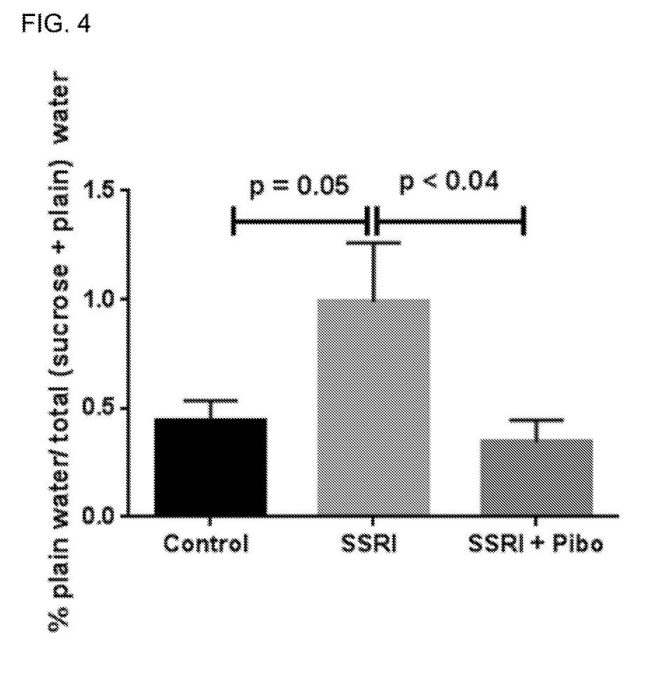

In the present invention, the serotonin-4 receptor (5-HT4R) has been identified as a critical player in SERT modulation, ENS and CNS development and GI function. Based on this data, the novel hypothesis that antagonism of the 5-HT4R would prevent the intestinal and behavioral defects caused by maternal SSRI exposure was tested. It was demonstrated that the 5-HT4R antagonist, Piboserod, administered during pregnancy and breastfeeding to mouse mothers concomitantly receiving the SSRI, Fluoxetine, have total intestinal transit and colonic motility that is comparable with control mice. (FIGS. 1-2). Similarly, in behavioral tests, mice exposed to Fluoxetine in utero and during breastfeeding had increased levels of anxiety (as measured by novelty-suppressed feeding, FIGS. 3A-3D) and depression (as measured by the sucrose preference test, FIG. 4). Mice exposed to Piboserod and Fluoxetine concomitantly were behaviorally comparable to control mice. Administration of a 5-HT4R antagonist during neurodevelopment thus corrects the long-lasting gastrointestinal motility and behavioral defects caused by developmental exposure to SSRls.

Given the widespread use of SSRIs in pregnancy and their resultant effects, it is imperative to delineate the mechanisms by which SSRIs cause abnormal GI and CNS development so that novel, targeted therapeutics can be designed to treat or prevent these abnormalities from occurring. The results of this study extend beyond patients with GI problems after developmental SSRI exposure to individuals with other conditions known to be caused or associated with SERT dysfunction including subsets of patients with irritable bowel syndrome, autism spectrum disorders, ulcerative colitis, microscopic colitis, or behavioral disorders such as depression and anxiety.

All patents, patent applications, and publications cited herein are incorporated herein by reference in their entirety as if recited in full herein.

The invention being thus described, it will be obvious that the same may be varied in many ways. Such variations are not to be regarded as a departure from the spirit and scope of the invention and all such modifications are intended to be included within the scope of the following claims.

1. A method of treating or preventing a disorder in a subject in need thereof, wherein the disorder is caused by in utero exposure and/or exposure through breast milk of the subject's mother to one or more agents that antagonize the serotonin reuptake transporter (SERT), comprising administering an effective amount of a 5-HT4 receptor antagonist to the subject.

2. The method of claim 1, wherein the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder, and combinations thereof.

3. The method of claim 2, wherein the gastrointestinal disorder is selected from the group consisting of: constipation, intestinal inflammatory disease, irritable bowel syndrome, enteric nervous system hyperplasia, Crohn's disease, ulcerative colitis, microscopic colitis, and combinations thereof.

4. The method of claim 1, wherein the one or more agents is selected from the group consisting of: a selective serotonin reuptake inhibitor (SSRI), a serotonin-norepinephrine reuptake inhibitor (SNRI), a trycyclic antidepressant (TCA), an atypical antidepressant, and combinations thereof.

5. The method of claim 4, wherein the agent is an SSRI.

6. The method of claim 1, wherein the subject is a mammal.

7. The method of claim 6, wherein the subject is human.

8. The method of claim 1, wherein the 5-HT4 receptor antagonist is administered to the subject's mother, while the subject is in utero.

9. The method of claim 8, wherein the 5-HT4 receptor antagonist is administered concomitantly with the one or more agents.

10. The method of claim 8, wherein the 5-HT4 receptor antagonist is administered perinatally.

11. The method of claim 1, wherein the 5-HT4 receptor antagonist is administered to the subject's mother and a therapeutically effective amount of the 5-HT4 receptor antagonist is passed to the subject through the subject's mother's breast milk.

12. The method of claim 1, wherein the 5-HT4 receptor antagonist is selected from the group consisting of: Piboserod, indisetron, fabesetron, sulamserod, E-2630, GR-113,808, GR-125,487, LY-297524, M-0014, R-216073, RO-1160367, RS-23597-190, RS-39604, SB-203,186, SB-204,070, SB-205,008, SB-205,800, SB-207,710, SER-103, SER-120, chamomile (ethanol extract), SDZ 205-557, and SC-53606.

13. The method of claim 11, wherein the 5-HT4 receptor antagonist is Piboserod.

14. A method of reducing the risk of developing a disorder in a subject, wherein the subject has been exposed in utero to a serotonin reuptake transporter (SERT) antagonist, comprising administering a therapeutically effective amount of a 5-HT4 receptor antagonist to the subject's mother during pregnancy.

15. The method of claim 14, wherein the disorder is selected from the group consisting of: a gastrointestinal disorder, a central nervous system disorder, an anxiety disorder, a mood disorder, a depressive disorder, an autism spectrum disorder, a substance abuse or dependence disorder, an attention deficit hyperactivity disorder, and combinations thereof.

16. The method of claim 15, wherein the gastrointestinal disorder is selected from the group consisting of: constipation, intestinal inflammatory disease, irritable bowel syndrome, enteric nervous system hyperplasia, Crohn's disease, ulcerative colitis, microscopic colitis, and combinations thereof.

17. The method of claim 14, wherein the SERT antagonist is a selective serotonin reuptake inhibitor (SSRI), a serotonin-norepinephrine reuptake inhibitor (SNRI), a trycyclic antidepressant (TCA), an atypical antidepressant, or combinations thereof.

18. The method of claim 17, wherein the SERT antagonist is an SSRI.

19. The method of claim 14, wherein the subject is a mammal.

20. The method of claim 19, wherein the subject is human.

21. The method of claim 14, wherein the 5-HT4 receptor antagonist is administered perinatally.

22. The method of claim 14, further comprising administering the 5-HT4 receptor antagonist to the subject postnatally.

23. The method of claim 22, wherein the 5-HT4 receptor antagonist is administered to the subject's mother and a therapeutically effective amount of the 5-HT4 receptor antagonist is passed to the subject through the subject's mother's breast milk.

24. The method of claim 14, wherein the 5-HT4 receptor antagonist is selected from the group consisting of: Piboserod, indisetron, fabesetron, sulamserod, E-2630, GR-113,808, GR-125,487, LY-297524, M-0014, R-216073, RO-1160367, RS-23597-190, RS-39604, SB-203,186, SB-204,070, SB-205,008, SB-205,800, SB-207,710, SER-103, SER-120, chamomile (ethanol extract), SDZ 205-557, and SC-53606.

25. The method of claim 24, wherein the 5-HT4 receptor antagonist is Piboserod.
